(19) United States
(12) Patent Application Publication
(10) Pub. No.: US 2026/0301891 A1
(43) Pub. Date: Oct. 1, 2026

(54) METHOD FOR VISUALLY PRESENTING MEDICAL TEST RESULTS IN A TIMELINE USED FOR ARTIFICIAL INTELLIGENCE ANALYSIS

(71) Applicants: TOPCON CORPORATION, Tokyo (JP); Topcon Healthcare, Inc., Oakland, NJ (US)

(72) Inventor: Hanna Kaarina KINNUNEN, Helsinki (FI)

(73) Assignees: TOPCON CORPORATION, Tokyo (JP); Topcon Healthcare, Inc., Oakland, NJ (US)

(21) Appl. No.: 19/095,335

(22) Filed: Mar. 31, 2025

(51) Int. Cl. G16H 10/40; G16H 10/60

(52) U.S. Cl. CPC G16H 10/40; G16H 10/60

(57) ABSTRACT

A method for viewing medical test results in a timeline includes the step of transmitting selected medical test results from a storage device to an AI function in response to activation of the AI function and receiving an AI analysis report based on the selected medical test results from the AI function. Thumbnails of the selected medical test results are displayed on a display along with medical test result icons displayed in a timeline on the display, the medical test result icons representing medical test results. An AI analysis report icon is also displayed in the timeline where the AI analysis report icon represents the AI analysis report.

### FIELD OF THE INVENTION

The present disclosure relates to a method for displaying medical test results analyzed by artificial intelligence (AI) in a timeline, and more particularly, to a method that allows a user to easily review and comprehend medical test results that are analyzed by AI by displaying icons related to medical test results on a timeline and displaying the medical test results.

### BACKGROUND

Medical test results are important sources of information for diagnosing and treating various diseases. However, medical test results are often complex and difficult to interpret, especially when they involve multiple parameters and time points. Moreover, medical test results may not be consistent or comparable across different laboratories, devices, or methods. Therefore, there is a need for a method that can simplify and standardize medical test results and make them more accessible, reviewable, and understandable for users.

### SUMMARY

A method for viewing medical test results in a timeline includes the steps of t21ransmitting selected medical test results from a storage device to an AI function in response to activation of the AI function and receiving an AI analysis report based on the selected medical test results from the AI function. Thumbnails of the selected medical test results are displayed on a display. Medical test result icons are also displayed in a timeline on the display with the medical test result icons representing medical test results. An AI analysis report icon is also displayed on the timeline with the AI analysis report icon representing the AI analysis report. In one embodiment, the medical test result icons comprise medical test result icons representing selected medical test results and medical test result icons representing unselected medical test results. The selected medical test results can be automatically selected or selected by a user. In one embodiment, the selected medical test results are automatically selected based on the selected medical test results being the latest medical test results available. In one embodiment, the medical test result icons representing selected medical test results are highlighted. In one embodiment, a second AI analysis report icon is displayed representing a second AI analysis report generated based on second selected medical test results different from the selected medical test results. In one embodiment, medical test result icons representing the second selected medical test results are highlighted in response to selection of the second AI analysis report icon.

An apparatus having memory storing computer program instructions for viewing medical test results in a timeline and a computer readable medium storing instructions for viewing medical test results in a timeline are also described herein.

### BRIEF DESCRIPTION OF THE DRAWINGS

FIG. 1 shows a clinical decision support system (CDSS) in communication with various examination devices according to one embodiment;

FIG. 2 shows the CDSS of FIG. 1 along with its configuration with respect to other hardware and software components of a patient health system;

FIG. 3 shows a high-level schematic of a computer for implementing method and systems described herein;

FIG. 4 shows a method for an eye care professional to use AI in order to assist in performing a patient's eye health exam;

FIG. 5 shows an image analysis data flow illustrating how data flows among a user, data management software, and an AI product according to one embodiment;

FIG. 6 shows an interface for viewing medical test results in a timeline according to one embodiment;

FIG. 7 shows a method for viewing medical test results in a timeline according to one embodiment;

FIG. 8 shows how the steps of the method shown in FIG. 6 are performed with respect to patient health system shown in FIG. 2;

FIG. 9 shows a process for activating an AI function according to one embodiment;

FIG. 10 shows a display according to one embodiment in which the medical test results that are to be analyzed by an AI function are identified;

FIG. 11 shows a display according to one embodiment in which past medical test results are used when more recent medical test results are not available;

FIG. 12 shows a display according to one embodiment in which it is determined that medical test results that are required for analysis by an AI function to generate a report are missing;

FIG. 13 shows a display according to one embodiment in which two reports have been generated and icons related to the reports are displayed;

FIG. 14 shows a display in which a past report is shown located before a current time according to one embodiment;

FIG. 15 shows how examination parameters are selected for analysis according to one embodiment;

FIG. 16 shows how an AI analysis tool menu is opened after selection by a user according to one embodiment;

FIG. 17 shows a popup window that is displayed after a user has selected an icon from the AI analysis tool menu;

FIG. 18 shows a popup window that is displayed after a user has confirmed the images to be analyzed and an AI analysis tool to be used for the analysis according to one embodiment;

FIG. 19 shows a popup window that is displayed after AI analysis is complete according to one embodiment;

FIG. 20 shows a thumbnail associated with reports that have been generated according to one embodiment;

FIG. 21 shows a report that was generated based on AI analysis according to one embodiment.

### DETAILED DESCRIPTION

One way to improve the usability of medical test results is to analyze the test results using artificial intelligence (AI) techniques (also referred to as AI or AI functions) to provide insights or recommendations. AI techniques can process large amounts of data and identify patterns, trends, anomalies, or correlations that may not be obvious to human experts. However, AI techniques may also have limitations or uncertainties, such as data quality, algorithm reliability, or ethical issues. Therefore, there is also a need for a method that can transparently and effectively communicate the results of AI analysis to users and allow them to verify, validate, or challenge the result or process of the AI analysis. The methods described herein allow a user to compare medical test results with AI analysis data and to examine the rationale, validity, and/or accuracy of the AI analysis. The methods described herein also improve the usability, transparency, and trustworthiness of medical test results and AI analysis.

In one embodiment, data management software comprises a plurality of modules for acquiring, storing, analyzing, and displaying various patient data. Each of the plurality of modules can be enabled or disabled for a particular entity or user of the data management software. The present disclosure describes a timeline function of data management software that includes a glaucoma clinical decision support system (CDSS), but the disclosure also supports timeline functions for other CDSSs for other types of medical conditions, medical issues, etc. In one embodiment, the CDSS is a module (i.e., a part of) the data management software and is used to estimate a patient's glaucoma risk score. The risk score is based on well-known algorithms. The purpose of the score, in one embodiment, is not to diagnose glaucoma but instead to help a user, such as a primary health care provider, to determine if there is a need for further examination or evaluation. In one embodiment, the algorithms that can be used for risk calculations include multi-factoral optical coherence tomography (OCT) screening score (MOS), glaucoma health score (GHS), and ocular hypertension treatment study (OHTS).

In one embodiment, each algorithm uses the patient's medical test results (also referred to as examination parameters) to calculate a risk score that will indicate the likelihood the patient will develop a glaucoma. The risk score can help a user make an informed decision regarding the patient's care and the next steps.

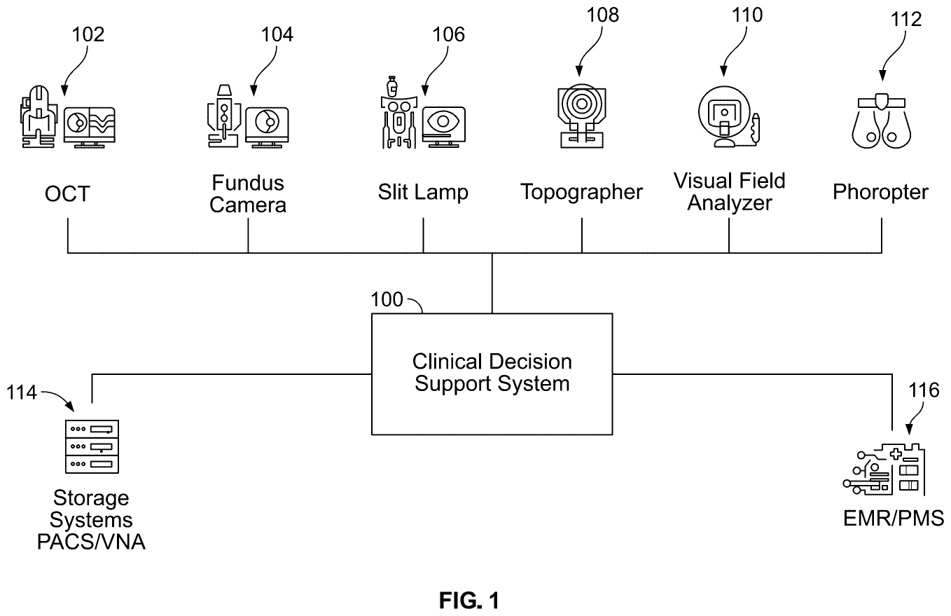

FIG. 1 shows a clinical decision support system (CDSS) 100 in communication with various examination devices including optical coherence tomography (OCT) device 102, fundus camera 104, slit lamp 106, topographer 108, visual field analyzer 110, and phoropter 112, all of which may be used to generate examination parameters during an examination of a patient's eye. CDSS 100 is also in communication with storage systems 114 comprising a picture archiving and communication system (PACS) and a vendor neutral archive (VNA). CDSS 100 is also in communication with electronic medical records (EMR) and practice management software (PMS) 116 which allows a user to access and update EMRs. Data received from examination devices 102-112 along with patient data from storage systems 114 and EMR/PMS 116 allows a user accessing CDSS 100 to display, review, and manage patient health data. CDSS 100 also assists a user in the selection and application of algorithms with respect to patient health data to determine a patient's condition and if that condition requires a referral to another doctor, such as a specialist. CDSS 100 can also be used for gathering and analyzing data in order to promote efficient and accurate decisions regarding a patient's health.

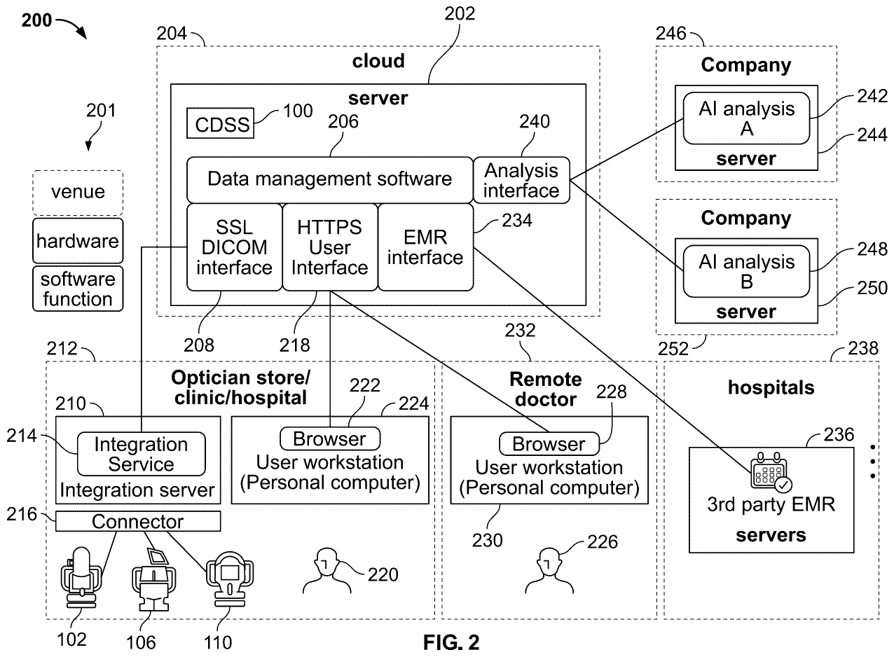

FIG. 2 shows CDSS 100 and its configuration and functionality with respect to other hardware and software in patient health system 200. It should be noted that a venue is identified in FIG. 2 by dashed line border, hardware is identified by a bold solid line border, and a software function is identified by a thin line border as shown by legend 201. CDSS 100 is implemented on server 202 which is implemented within cloud computing system 204. CDSS 100 is a module of data management software 206 which is also implemented on server 202. Data management software 206 is in communication with SSL DICOM interface 208, HTTPS user interface 218, EMR interface 234, and analysis interface 240, all of which are implemented on server 202. In one embodiment, CDSS 100, SSL DICOM interface 208, HTTPS user interface 218, EMR interface 234, and analysis interface 240 are implemented in data management software 206, however one or more of CDSS 100, SSL DICOM interface 208, HTTPS user interface 218, EMR interface 234, and analysis interface 240 can be implemented on server 202 as standalone modules. SSL DICOM interface 208 is in communication with integration server 210 located at an optician store 212 (which alternatively may be a clinic or hospital or any other location at which a patient may be evaluated).

Integration server 210 operates integration service 214 which receives data from devices including OCT device 102, slit lamp 106, and visual field analyzer 110 via connector 216. It should be noted that integration service 214 can receive data from additional devices, such as those shown in FIG. 1, although only three devices are shown in FIG. 2 for clarity.

HTTPS user interface 218 allows user 220 located at optician store 212 to access data using browser 222 operated on user workstation 224 (also referred to as personal computer) which are also located at optician store 212. HTTPS user interface 218 also allows user 226 located at remote doctor location 232 to access data using browser 228 operated on user workstation 230 (e.g., browser 228 and workstation 230 together form a client and the client can include/support other types of software) which are also located at remote doctor location 232.

Electronic medical record (EMR) interface 234 is implemented on server 202 and allows data management software 206 to communicate with 3^{rd} party EMR implemented on 3^{rd} party EMR servers 236 operated at hospitals 238 (or the like). In one embodiment, EMRs are used for data exchange. For example, a remote EMR, such as 3^{rd} party EMR communicates with data management software 206. EMRs can also be sent to other doctors in order to obtain additional opinions.

In one embodiment, CDSS 100 has analysis software (e.g., artificial intelligence analysis software) and can analyze images and other patient data using artificial intelligence or other types of analysis software. CDSS 100 can also transmit information to other locations for analysis using artificial intelligence or other types of analysis software. Analysis interface 240 is implemented on server 202 and allows data management software 206 to communicate with company A 246 and company B 252. Company A server 244 implements Artificial Intelligence (AI) analysis algorithm A 242 and is used to analyze health data of a patient using a particular AI algorithm. Similarly, company B server 250 implements AI analysis algorithm B 248 and is used to analyze health data of a patient using a different AI algorithm. It should be noted that although company A 246 and company B 252 are described as implementing AI algorithms, other types of algorithms may be used as well to analyze health data of a patient.

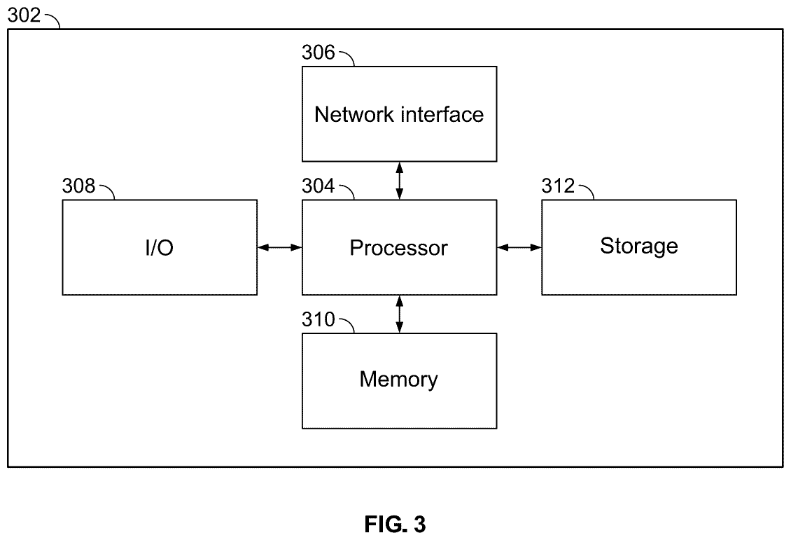

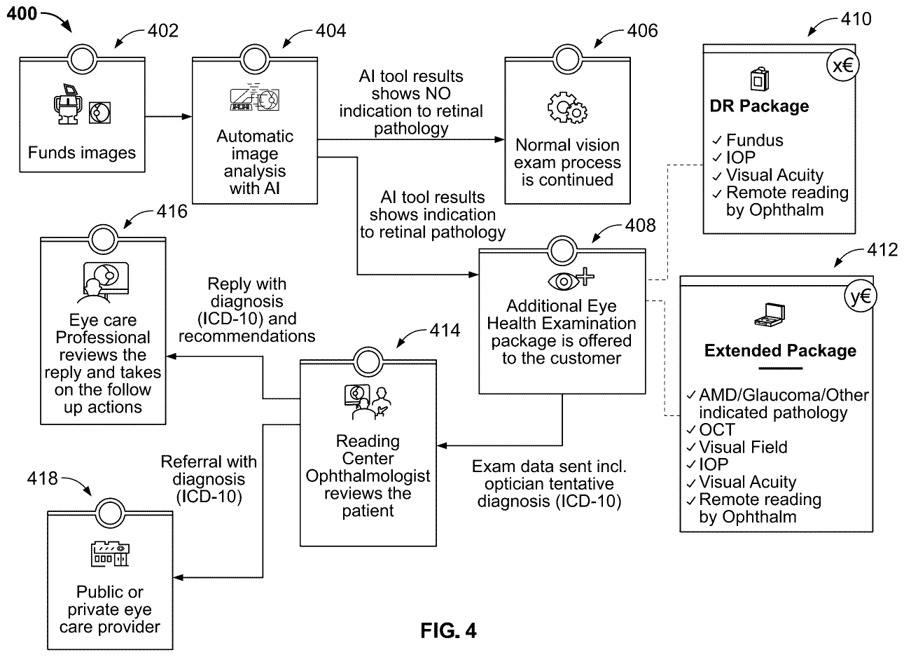

CDSS 100 shown in FIG. 1, as well as other devices described herein, can be implemented using one or more computers. In addition, the hardware identified in FIG. 2 by a bold solid line border as shown in legend 201 of FIG. 2 can also be implemented using one or more computers. A high-level block diagram of such a computer is illustrated in FIG. 3. Computer 302 contains a processor 804 which controls the overall operation of the computer 302 by executing computer program instructions which define such operation. The computer program instructions may be stored in a storage device 312, or other computer readable medium (e.g., magnetic disk, CD ROM, etc.), and loaded into memory 310 when execution of the computer program instructions is desired. Thus, the method steps of FIGS. 4, 5, and 7, the software functions (see legend 201) of FIG. 2, as well as other methods and algorithms described herein, can be defined by the computer program instructions stored in the memory 310 and/or storage 312 and controlled by the processor 804 executing the computer program instructions. For example, the computer program instructions can be implemented as computer executable code programmed by one skilled in the art. Accordingly, by executing the computer program instructions, the processor 304 executes an algorithm defined by the method steps of FIGS. 4, 5, and 7, the software functions of FIG. 2, or other methods and algorithms described herein. The computer 302 also includes one or more network interfaces 306 for communicating with other devices via a network. The computer 302 also includes input/output devices 308 that enable user interaction with the computer 302 (e.g., display, keyboard, mouse, speakers, buttons, etc.) In one embodiment, CDSS 100 requires the use of discrete data storage (DDS) to safely and securely store data. One skilled in the art will recognize that an implementation of an actual computer could contain other components as well, and that FIG. 3 is a high-level representation of some of the components of such a computer for illustrative purposes.

In one embodiment, CDSS 100 is part of data management software 206 and CDSS is opened (i.e., launched) from data management software 206. CDSS 100 is typically launched when a user (e.g., eye care professional) meets a patient who qualifies for a glaucoma screening. In one embodiment, a patient qualifies if, for example, they show symptoms or if the screening is part of a routine health assessment. The user then checks (e.g., reviews) the risk score of an algorithm that is currently active (i.e., selected). Risk scores of other algorithms can be checked as well. A user can then check medial test results (e.g., examination parameters) included in the calculation of risk scores. A user can change or exclude data where necessary and check the risk scores again.

In one embodiment, CDSS 100 cannot be launched before the user is logged into data management software 206. The user must also have a patient's information to open a clinical viewer, CDSS 100 must be enabled and the user must have a valid license for the CDSS. A user can then launch CDSS 100 by selecting a glaucoma tab in data management software 206. The dashboard launches and automatically shows a risk score for the patient immediately, calculated for the active algorithm using the latest examination results. In one embodiment, the active algorithm can be selected based on user or organizational preferences.

In one embodiment, CDSS 100 must have at least one algorithm enabled. Depending on configuration, several algorithms may be enabled and available for risk calculations. The active algorithm is the one currently selected in an algorithm view. The other algorithms with licenses are shown as individual tabs. They are referred to as the enabled algorithms.

If more than one algorithm is enabled, a user can change any one of the enabled algorithms to be the active algorithm and view the associated risk score. In one embodiment, each algorithm uses a set of different risk factors to calculate the risk score.

In one embodiment, for each active algorithm, an associated risk score is presented in numerical format. The risk score is also visualized with a graphic, such as a gauge or bar graph, where different color risk levels aid a user in reviewing the risk estimation. In one embodiment, individual risk factors associated with a risk score are displayed in addition to a risk score.

In one embodiment, CDSS 100 will automatically select the latest available medical test results to be used in risk calculations. A user can select medical test results (e.g., examination parameters) from a drop-down menu that shows all the available medical test results within a time range. In one embodiment, to change the medical test results used in the risk calculations a user can select medical test results for a selected eye (i.e., an eye of a patient that has been examined). After a user selects different medical test results, the risk score value and the graphic are updated based on the selected medical test results. It should be noted that, in one embodiment, fundus and OCT are interconnected. Changing the fundus image will also change the OCT, and vice versa. If a different algorithm is selected to analyze medical test results after different medical test results have been selected, the newly selected medical test results are analyzed using the newly selected algorithm.

It should be noted that bad or incorrect medical test results can be worse than no medical test results at all. Medical test results can be excluded if a user does not want to include them in the risk score calculation. In one embodiment, the risk score is recalculated after the user selects medical test results that should be excluded from the risk score calculation. If there are not enough medical test results to calculate a risk score after bad or incorrect medical test results are excluded, a risk score will not be calculated.

The benefits of CDSS 100 include better quality and efficiency of glaucoma testing and services, possible reimbursement for additional testing due to CDSS result indicating medical necessity, reduced medical liability, better efficiency of decision making, better quality of referrals, reduction of over-referrals, and higher patient retention rate.

The term artificial intelligence (AI), as used herein, pertains to any technique that enables computers to mimic human intelligence using logic, if-then rules, decision trees, and/or machine learning (including deep learning). Machine learning as used herein pertains to a subset of AI that includes statistical techniques that enable machines to improve at tasks with experience. Deep learning as used herein pertains to a subset of machine learning comprising algorithms that permit software to perform tasks, such as speech and image recognition, by using multi-layered neural networks to analyze vast amounts of data.

In one embodiment, AI is used in screening as follows. A patient is registered (e.g., an electronic medical record for the patient is generated in practice management software such as practice management software 116 of FIG. 1). Data management software 206 identifies the electronic medical record of the patient and creates worklists. Examinations are performed according to the worklists and the examination parameters generated during the examinations are stored in data management software 206. Images are sent for AI based image analysis (e.g., sent to company A 246 and/or company B 252 for analysis). The examination parameters and AI analysis are reviewed at a screening center. Based on the review, a patient may be referred to an ophthalmologist at a reading center. The ophthalmologist at the reading center reviews patient data including the examination parameters and AI analysis and replies to the screening center if needed. The ophthalmologist at the reading center can also refer the patient to an eye care provider. A referral, if needed is sent to the eye care provider along with relevant patient data. The screening center receives diagnosis, recommendations, and follow up instructions. In one embodiment, screening centers are healthcare facilities where initial examinations and tests are conducted to detect potential health issues in asymptomatic individuals. The primary goal is early detection of diseases or conditions to enable timely intervention and treatment. Reading centers, also known as diagnostic reading centers, are specialized facilities where medical images and test results are interpreted by trained professionals, typically radiologists or other specialists. These centers focus on analyzing and providing detailed reports on diagnostic images and data. Screening centers focus on early detection and prevention, and serve a wide range of populations in order to detect potential health problems as early as possible. Reading centers specialize in detailed analysis and interpretation of diagnostic data, often after initial screening has indicated a need for further investigation.

In one embodiment, AI can be used by an eye care professional performing eye health exams and assist the eye care professional in providing a patient with options for further examinations based on the results of the patient's eye health exam. FIG. 4 shows a method 400 for an eye care professional to use AI in order to assist in performing a patient's eye health exam. At step 402, fundus images are generated by examination equipment. At step 404, automatic image analysis is performed using AI. If the AI analysis results show no indication of retinal pathology, then the method proceeds to step 406 where the normal vision exam process continues. If the AI analysis results show an indication of retinal pathology, the method proceeds to step 408 and an additional eye health examination package is offered to the patient. For example, package 410 including exams pertaining to fundus, IOP, visual acuity, and remote reading in various tests (e.g., refraction, visual field, color vision, fundus examination, fundus three-dimensional image analysis (OCT), corneal shape, intracorneal capsular examination, deep vision, contrast examination, slit-lamp microscopy, intraocular pressure test, etc.) may be offered to the patient. Extended package 412 includes exams pertaining to AMD/glaucoma/other indicated pathology, OCT, visual field, IOP, visual acuity, and remote reading in the various tests may also be offered to the patient. Examination parameters/data and an eye care professional's tentative diagnosis are then sent to a reading center as shown in step 414 where an ophthalmologist reviews the examination parameters/data and other patient information. The ophthalmologist can reply with a diagnosis and recommendations which, at step 416, the eye care professional can review and perform follow up examinations and/or actions. The ophthalmologist can alternatively reply with a referral to another public or private eye care provider (e.g., another ophthalmologist) as shown in step 418 along with a diagnosis.

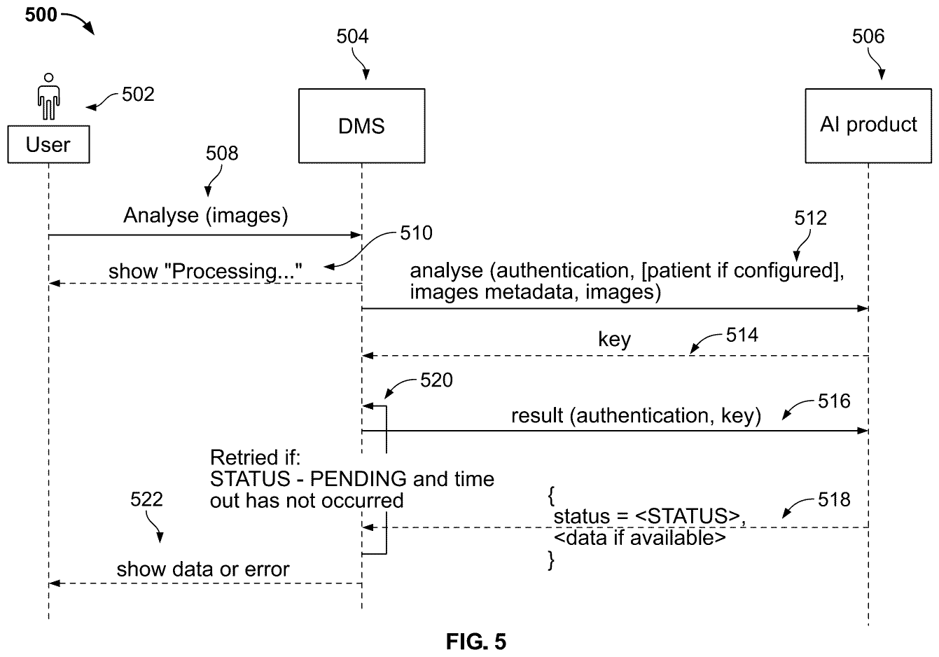

FIG. 5 shows data flow 500 for image analysis indicating how data flows among user 502, data management software 504 (shown as DMS in FIG. 5), and AI product 506 (e.g., AI that is used to analyze images and/or medical test results). User 502 transmits request 508 to analyze images to data management software 504. In response, data management software 504 transmits command 510 to display “Processing . . . ” to user 502. Data management software 504 transmits request 512 to AI product 506 to analyze images in response to request 508. Request 512 includes authentication data, patient information (if data management software 504 is configured to send patient information), image metadata, and images. AI product 506, transmits key 514 to data management software 504. In response to receipt of key 514, data management software 504 transmits result 516 including authentication and key 514. In response to receipt of the result 516, AI product 506 transmits the status of its operation and data that results from the analysis 518 if available. If a result is not available, the data flow returns 520 to repeat transmission of result 516. In response to receipt of data that results from the analysis 518, the data that results from the analysis 518 is transmitted to user 502 or an error message, if there was a problem with the analysis.

The AI analysis provided by AI product 506 of FIG. 5 can perform various functions. AI can be used to analyze a fundus image in order to recognize diseases such as diabetic retinopathy (DR), age related macular degeneration (AMD), and glaucoma and generate a severity classification. AI can also be used to analyze an OCT scan in order to recognize pathologies and generate an indication of their severity. AI can further be used for multi-modal analysis of images and scans, other eye data such as perimetry or intra-ocular pressure (IOP), and a patient's account of their medical history (i.e., anamnesis). AI can also be used for multi-source analysis and forecasting based on various exam data and history, patient history, normative data, different AIs, etc. AI can produce outcomes including diagnosis, recommendations, and clinical guidance. AI analysis can help eye care professionals, eye care practitioners, opticians, and/or optometrists make informed decisions about which patients to send to an ophthalmologist for review, assist in early detection of diseases, and help both optometrists and ophthalmologists provide more services to patients.

Images can be sent for analysis in one of two ways. First, images can be sent for analysis on demand. In this case, a user can select the images to be sent and then select a button that causes the selected images to be sent for analysis. Second, a set of rules can be configured to automatically send image for analysis based on one or more factors such as the type of device used to generate the images, etc. In either case, when an AI analysis is complete, the user is notified. In one embodiment, a pop-up window will appear with results of the AI analysis. A report may also be generated including the results of the AI analysis. Regulatory information can be included in analysis results and/or reports for convenience. In one embodiment, AI usage is monitored and reports regarding AI usage can be generated for review.

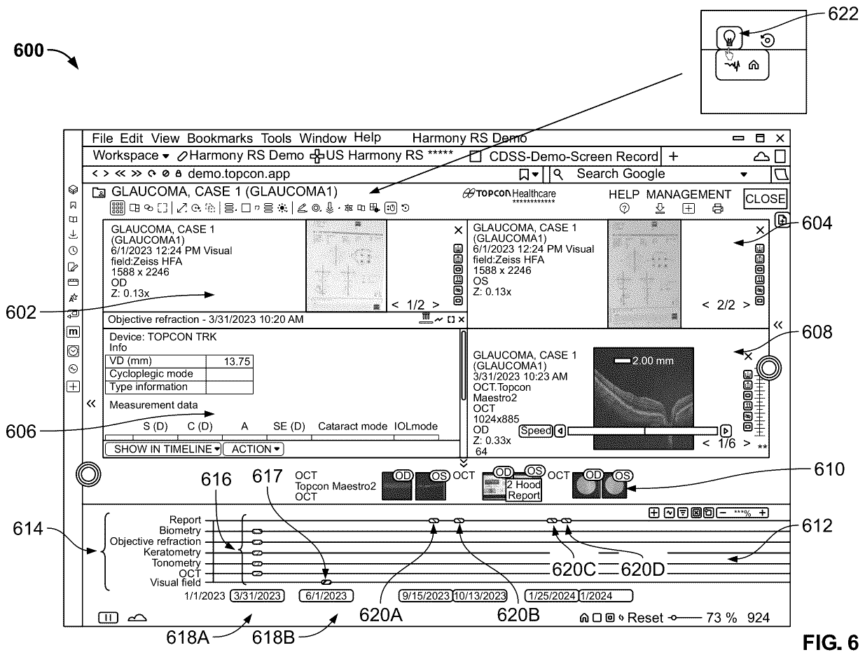

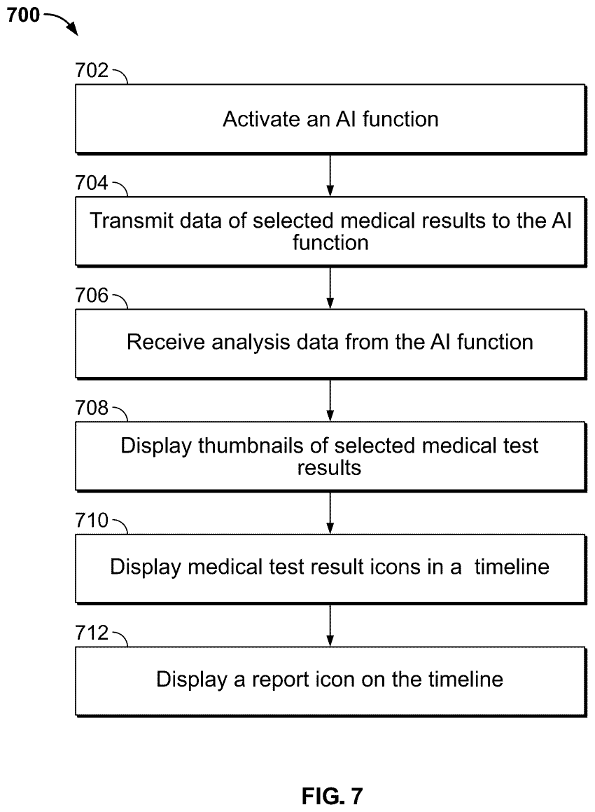

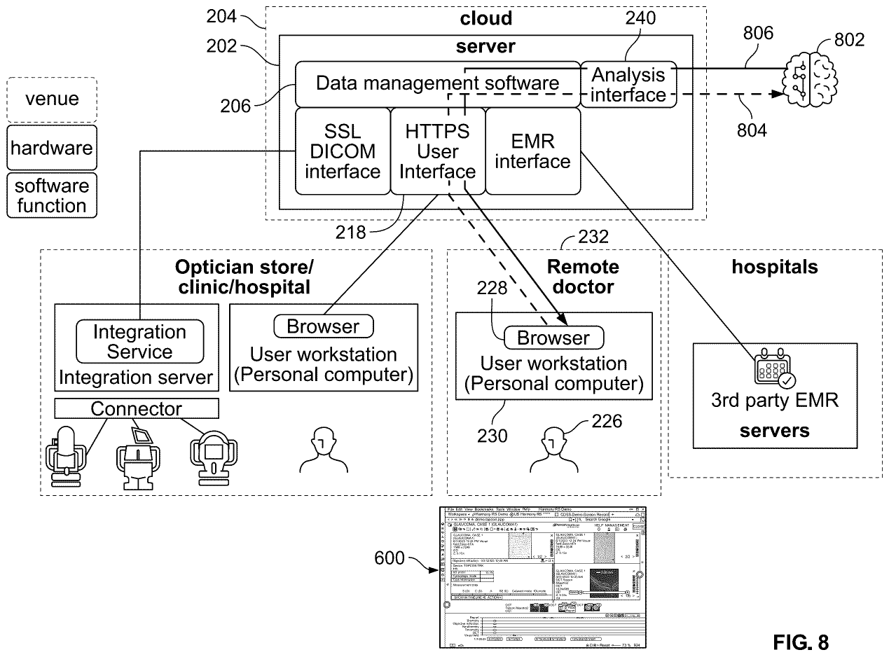

In one embodiment, an interface shows medical test results, a timeline having icons representing medical test results and reports, and thumbnails of medical test results that have been selected for analysis by an AI function. FIG. 6 shows interface 600, according to one embodiment, displaying medical test results in an upper portion of interface 600, thumbnails of medical test results shown in the lower center of interface 600, and a timeline 610 shown at the bottom of interface 600. Medical test results 602, 604, 606, and 608 are shown in a 2 by 2 matrix according to one embodiment. In one embodiment, medical test results 602, 604, 606, and 608 have been selected for analysis by an AI function. FIG. 6 also shows medical test result thumbnails 610 which comprise thumbnail images of medical test results and/or other information selected from timeline 612. It should be noted that data (e.g., medical test results/examination parameters) to be analyzed using an AI function can be selected from the 2 by 2 matrix or timeline 612. Timeline 612 comprises a vertical axis and horizontal axis. The vertical axis of timeline 612 comprises a plurality of horizontal lines having labels 614 which identify what the icons on each horizontal line represent. In this embodiment, labels 614 identify icons located on each of the horizontal lines, from top to bottom, as a Report, a Biometry medical test result, an Objective Refraction medical test result, a Keratometry medical test result, a Tonometry medical test result, an OCT medical test result, and a Visual Field medical test result. The horizontal axis of timeline 612 represents a timeline and indicates dates on which the medical tests were performed and reports were generated. Earlier dates are shown on the left side of timeline 612 and later dates are shown on the right side of timeline 612. Dates 618A and 618B are shown below the horizontal lines and indicate dates associated with the medical test result icons shown above the dates. Medical test result icons 616 and 617 are shown displayed on horizontal lines of timeline 612 and the position of each test result icon represents the date on which the test result was generated. Each icon shown on timeline 612 can be selected to display the associated medical test result (or report). Report icons 620A-620D are shown displayed on horizontal lines of timeline 612 and the position of each report icon represents the date on which the report was generated. In one embodiment, interface 600 has activate AI icon 622 which a user can select in order to activate an AI function (described in further detail below in connection with FIGS. 7 and 8).

FIG. 7 shows a flowchart of method 700 for generating reports based on medical test results and displaying related information. Method 700 of FIG. 7 is described in connection with patient health system 200 shown in FIG. 8. It should be noted that some labels shown in FIG. 2 have been omitted from FIG. 7 for clarity. In addition, company A 246 and company B 252 which provide AI analysis shown in FIG. 2 are shown in FIG. 7 as 3^{rd} party AI tools 702. As shown in FIG. 7, method 700 starts at step 702 in which an AI function is activated. In one embodiment, user 226, shown in FIG. 8, interacts with interface 600 via browser 228 of user workstation 230 located at remote doctor 232 location. The activation of AI by user 226 at step 702 is transmitted from workstation 230 through HTTP user interface 218 to data management software 206 as shown by arrow 804. At step 704 of method 700, data management software 206 transmits selected medical test results through analysis interface 240 to 3^{rd} party AI tools 802. 3^{rd} party AI tools 802 (i.e., the AI function) analyze the selected medical test results and sends the result of the analysis to data management software 206 via analysis interface 240 as shown by arrow 806. Data management software 206 receives an AI analysis report from 3^{rd} party AI tools 802 (i.e., the AI function) as shown in step 706 of method 700 of FIG. 7. The AI analysis report is based on the selected medical test results. Data management software 206 then transmits the AI analysis report to user workstation 230 for display to user 226 via browser 228. At step 708, thumbnails 610 of the selected medical test results are displayed on interface 600. At step 710, medical test result icons 616 and 617 are displayed on timeline 612. At step 712, an AI analysis report icon (i.e., one of report icons 620A-620D) is displayed on timeline 612. The AI analysis report icon displayed at step 712 represents the AI analysis report. In one embodiment, the AI analysis report is displayed in the 2 by 2 matrix shown in FIG. 6 but the AI analysis report can be displayed using other display methods as well. In one embodiment, the medical test result icons comprise medical test result icons representing selected medical test results and medical test result icons representing unselected medical test results. The selected medical test results can be automatically selected or selected by a user. In one embodiment, the selected medical test results are automatically selected based on the selected medical test results being the latest medical test results available. In one embodiment, the medical test result icons representing selected medical test results are highlighted. In one embodiment, a second AI analysis report icon is displayed representing a second AI analysis report generated based on second selected medical test results different from the selected medical test results. In one embodiment, medical test result icons representing the second selected medical test results are highlighted in response to selection of the second AI analysis report icon.

The AI function can be any algorithm or model that can process medical test results and provide AI analysis data, such as insights, recommendations, predictions, or classifications. The AI function can be activated by a user's command, a predetermined condition, and/or a scheduled time. Visual highlighting of selected medical test results icons can be accomplished using different colors, shapes, sizes, fonts, or symbols, or by separating, enlarging, or zooming in on the data of the medical test results that are used for the AI analysis. The AI function used can be selected by a user via interface 600, selected by default, or selected based on medical test results to be analyzed. In one embodiment, the medical test result thumbnails display selected medical test results as indicated in step 708 of method 700.

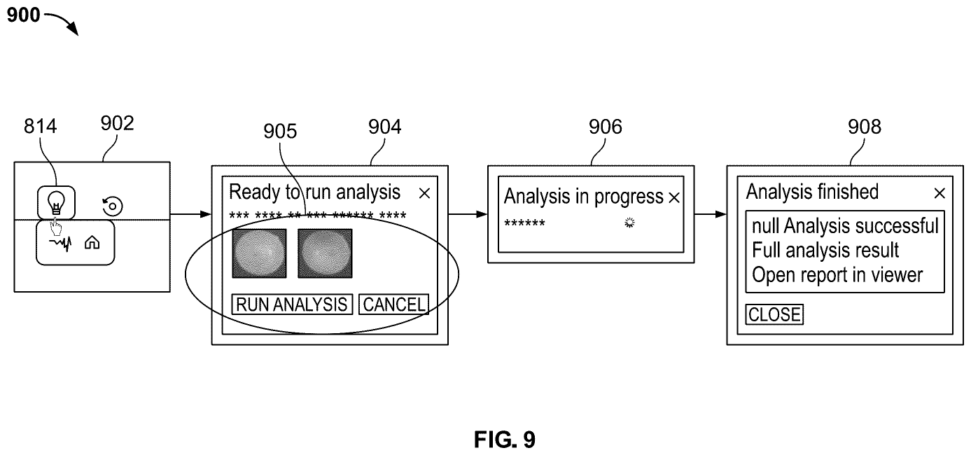

FIG. 9 shows screen flow 900 for activating an AI function and confirming the medical test results to be analyzed by the AI function. At screen 902, a user selects activate AI icon 814. In response, at screen 904, medical test results 905 that will be analyzed by the AI function are displayed. In one embodiment, a user can choose to accept the displayed test results or choose other medical test results for analysis instead by unselecting the currently selected medical test results (e.g., clicking on the medical test result icon) and selecting other medical test results (e.g., by clicking on the medical test result icon). At screen 906, text indicating that the analysis is in process is displayed to a user until screen 908 at which text indicated that the analysis is complete is displayed to the user.

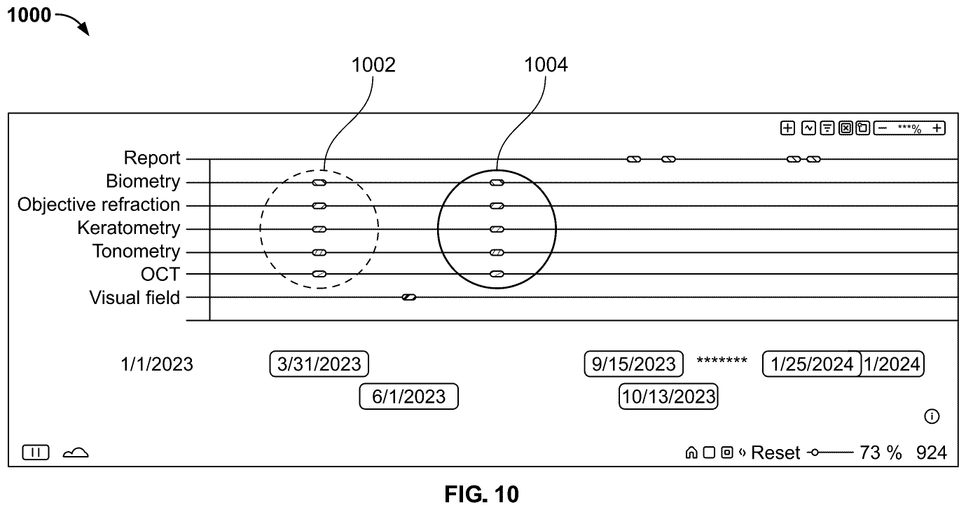

FIGS. 10-14 show various operations related to selection of medical test results and analysis of medical test results.

FIG. 10 shows timeline 1000 according to one embodiment in which the medical test results analyzed by an AI function are identified. Medical test results 1002 that are analyzed by an AI function are shown highlighted and/or blinking on timeline 1000. Medical test result 1002 is initially selected as the subject to be analyzed by the AI function. If this selected medical test result 1002 is not appropriate, or if the user wishes to select another medical test result, the user can select it with a pointing device such as a mouse. For example, medical test results 1004 can instead be selected by a user to be analyzed and medical test results 1004 are shown not highlighted or blinking. Once the user selects medical test result 1004, medical test result 1002 is no longer highlighted and or blinking, and instead medical test result 1004 is highlighted and or blinking.

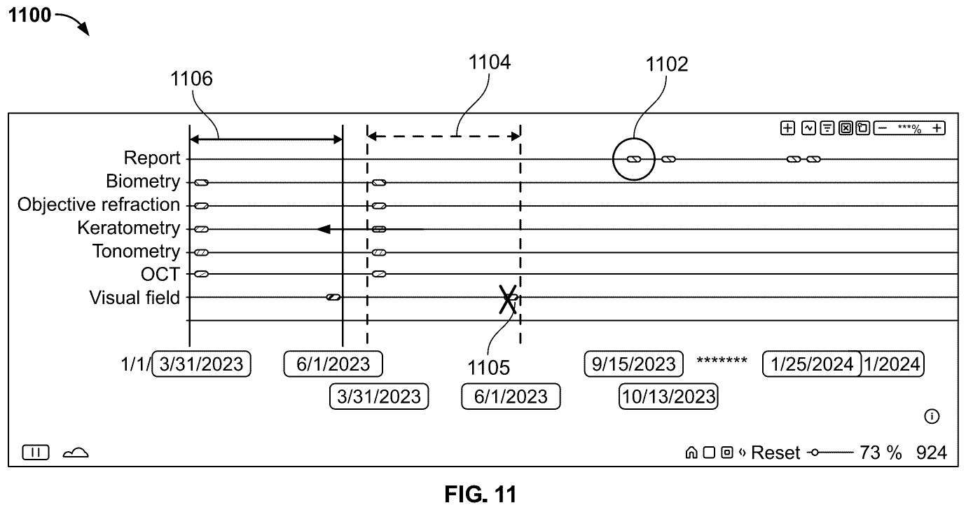

FIG. 11 shows timeline 1100 according to one embodiment in which past medical test results (in a period of time) are analyzed by the AI function when more recent medical test results are not available. Report 1102 would normally be generated by AI function analysis of medical test results 1104 which are more recent than medical test results 1106. However, if a required medical test result is missing, such as medical test report 1105 shown crossed out, past (i.e., earlier) medical test results 1106 may be selected for analysis by an AI function instead. When analyzing medical test results as report 1102, medical test results 1104 from the period in which the most recent medical test results are initially available or could be selected. However, medical test result 1104 is missing one of the test results originally required for analysis, the test result 1105. In this case, medical test result 1106, a period of time when sufficient medical test results are available, is automatically selected or selected by the user. This allows for an analysis that meets the required medical test results, which can then be visually confirmed.

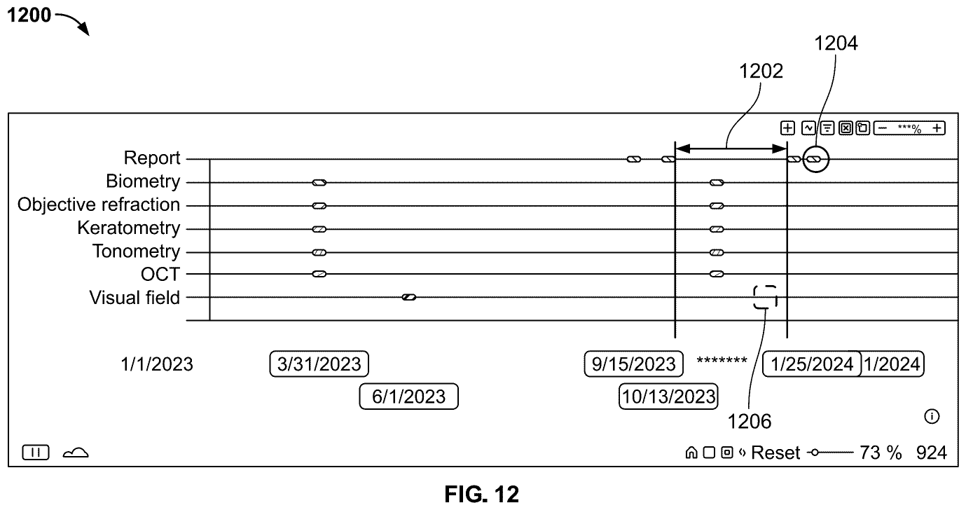

FIG. 12 shows timeline 1200 according to one embodiment in which it is determined that medical test results that are required for analysis by an AI function to generate a report are missing. As shown in FIG. 12, one medical test result 1206 of medical test results 1202 that are required by an AI function to generate report 1204 is missing. As such, the AI function cannot be activated and a user will be informed of that by a notice such as text in a pop-up window. Alternatively, a frame line indicating a missing 1206 position or line on the medical test result to make the user aware that the test result is missing.

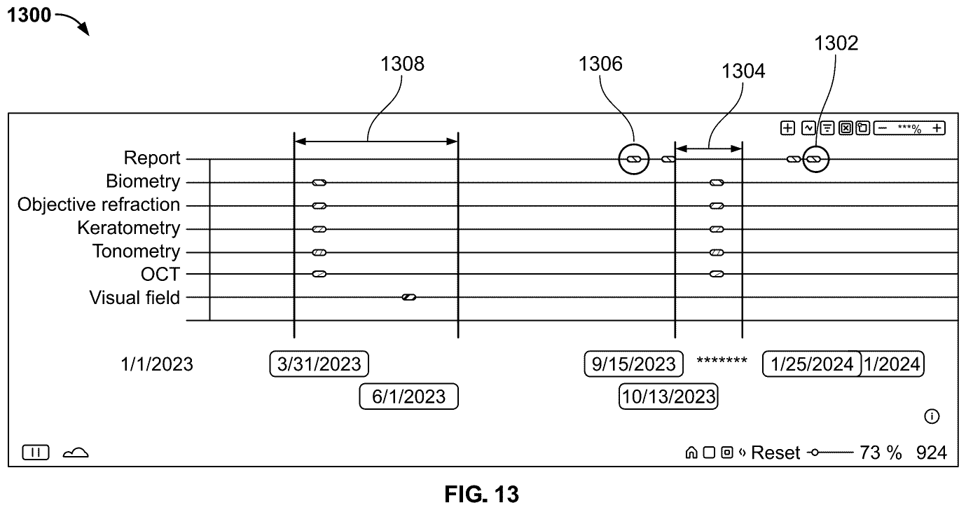

FIG. 13 shows timeline 1300 according to one embodiment in which two reports can be generated. Current report 1302 is generated by activating an AI function to analyze current medical test results 1304. Past report 1306 is generated by activating an AI function to analyze past medical test results 1308 which are older than current medical test results 1304. By comparing report 1302 and report 1306, one can see the changes in the report at the time of medical test result 1308 and at the time of medical test result 1304 at different times.

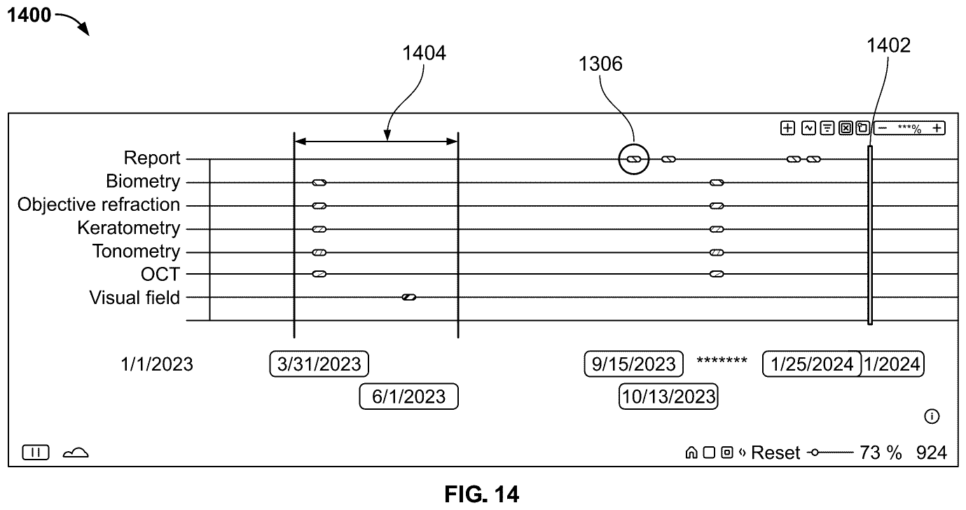

FIG. 14 shows timeline 1400 in which past report 1306 is shown located before current time 1402 so that a user looking at display 1400 can determine that past report 1306 is based on medical test results acquired at an earlier point in time. The system is configured to allow a user to create a report based on the past medical test result 1404 and to place the report in a past position. When the user selects the past medical test results 1404 to create past report 1306 that is the result of analysis by the AI function, the past report 1306 can be placed in a past position on the timeline that is not the current position 1402.

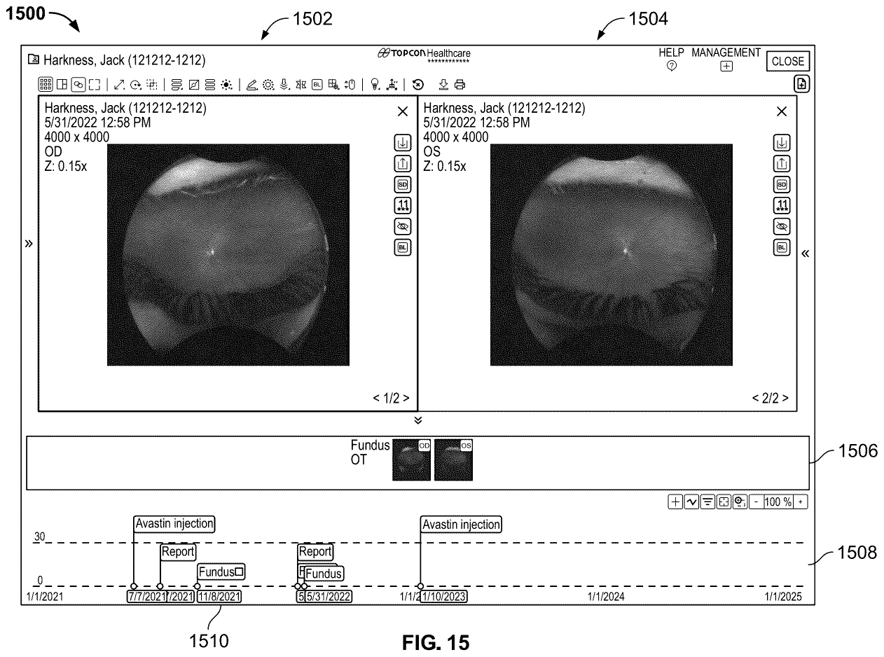

FIGS. 15-21 show how examination parameters are selected for analysis by AI. FIG. 15 shows display 1500 in which a user is selecting examination parameters (in this case, images) to be analyzed by AI. Image 1502 and image 1504 are shown located in a viewing area above thumbnail display 1506 and timeline 1508. In this embodiment, icons shown in timeline 1508 have flags, such as flag 1510 having the text “Fundus”. Flags can be used to identify icons on timeline 1508. Timeline 1508 as shown in FIG. 15 is expanded and flags can be used to identify icons. In one embodiment, timeline 1508 is collapsed to show only icons. In one embodiment, timeline 1508 can be collapsed completely in order to provide a user with a larger view of images 1502 and 1504. Images 1502 and 1504 can be selected either by selecting an icon from timeline 1508 or by selecting one or more thumbnails from thumbnail display 1506. Collapsing and expanding timeline 1508 can cause icons to be displayed instead of flags depending on how much room is available to display information in timeline 1508. In one embodiment, a user can add flags and/or icons in order to identify when certain medical actions have occurred such as treatments provided and/or administration of medicine. In one embodiment, thumbnails in thumbnail display 1506 can be selected for viewing in a larger image. In one embodiment, clicking a flag or icon will display information associated with the flag or icon. For example, clicking on a report flag or icon can show thumbnails or full-size images of images, examination parameters, or reports associated with the selected flag or icon.

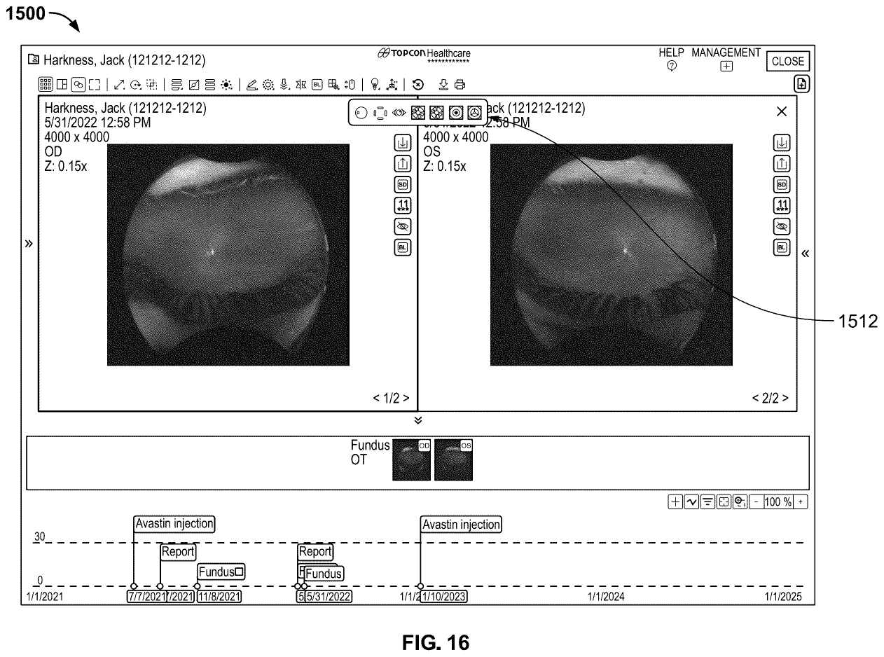

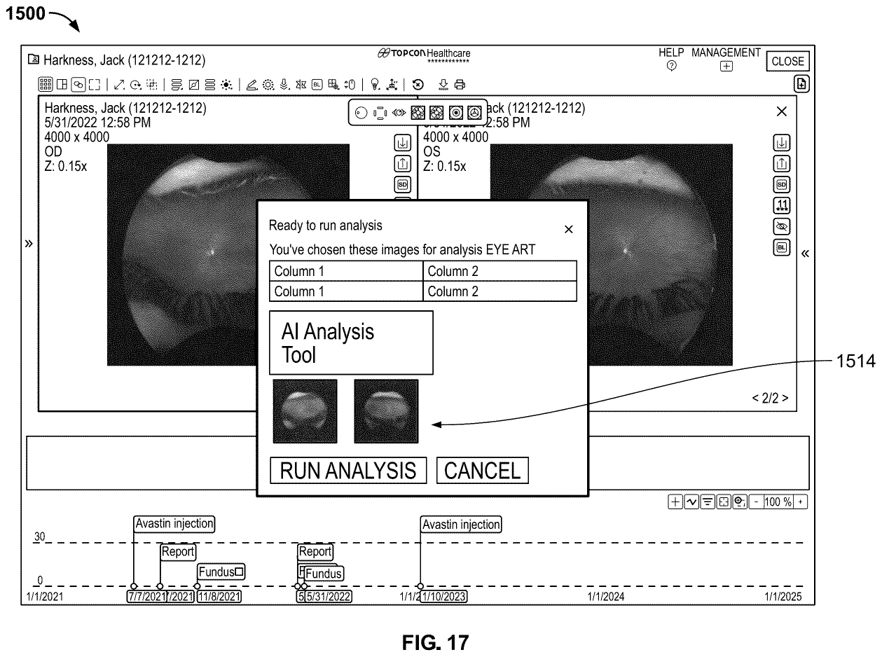

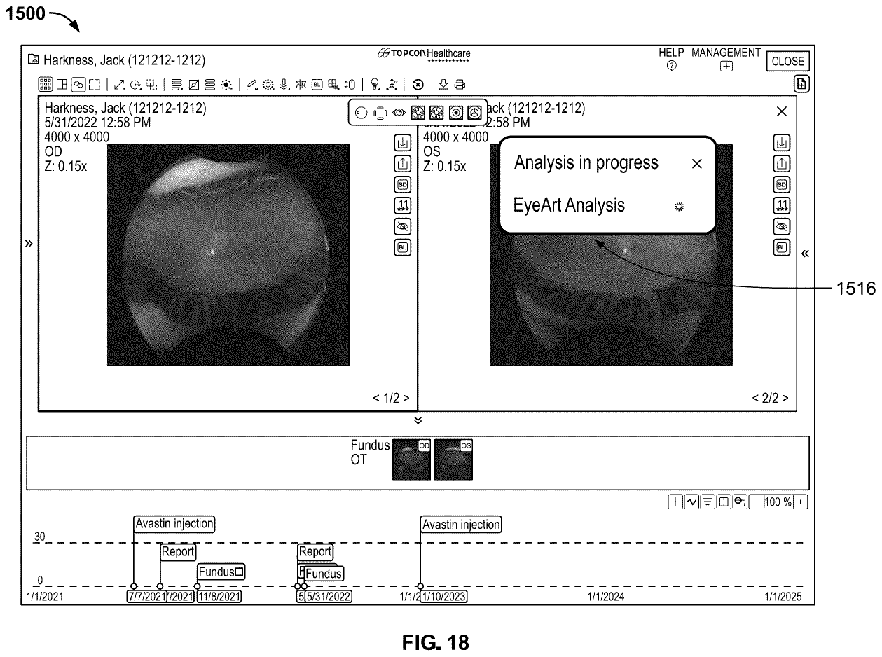

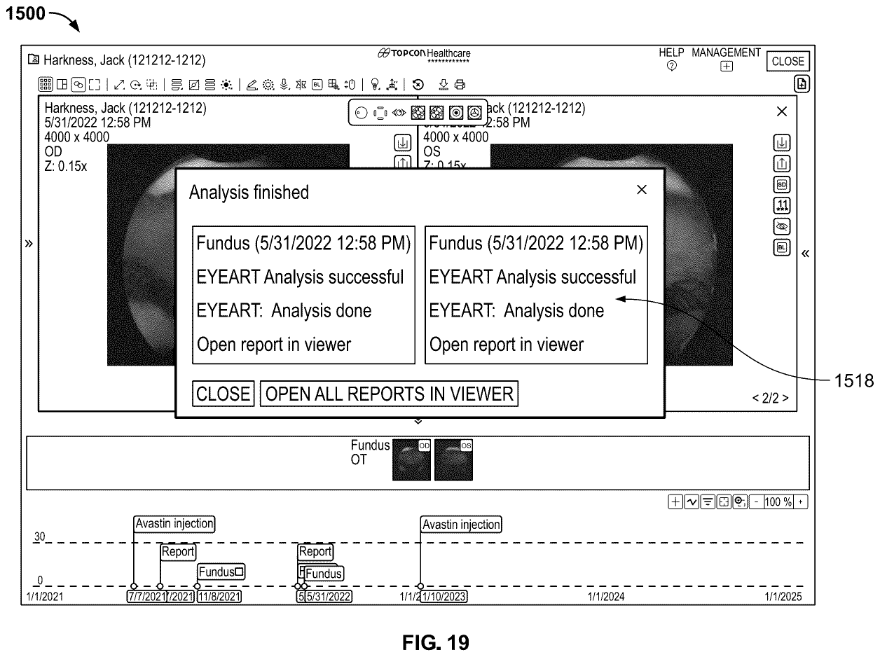

FIG. 16 shows a user having opened AI analysis tool menu 1512 from which the user can choose how examination parameters (e.g., images) can be analyzed. FIG. 17 shows popup window 1514 which is displayed after a user has selected an icon from AI analysis tool menu 1512 to have AI analyze images. Pop up window 1514 also shows thumbnails of images that have been selected for analysis by an AI analysis tool. In one embodiment, popup window 1514 display thumbnails of images that have been selected for analysis by an AI analysis tool. A user can cancel the proposed analysis of images by a particular AI analysis tool or choose to run the analysis of images by the particular AI analysis tool. FIG. 18 shows popup window 1516 which is displayed after a user has confirmed the images to be analyzed and the AI analysis tool to be used for the analysis. Popup window 1516 indicates that the analysis is in progress and can display a progress graphic to show that the AI analysis is being performed and how much longer the analysis will take to complete. FIG. 19 shows popup window 1518 which is displayed after AI analysis has been completed. Popup window 1518, in one embodiment, shows details of one or more reports created based on AI analysis. In one embodiment, a user is offered an option to view all generated reports in a viewer by selecting a button shown in popup window 1518.

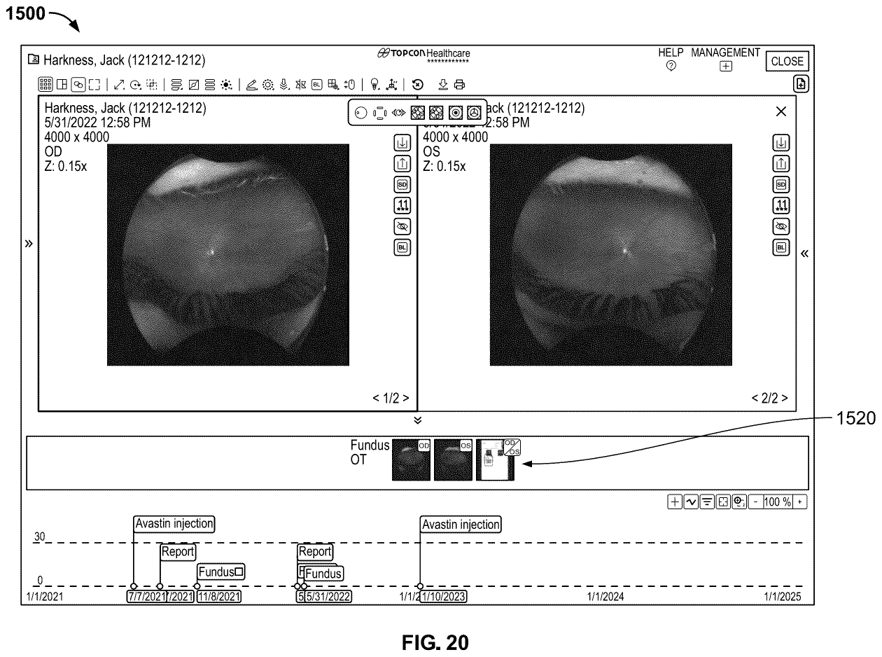

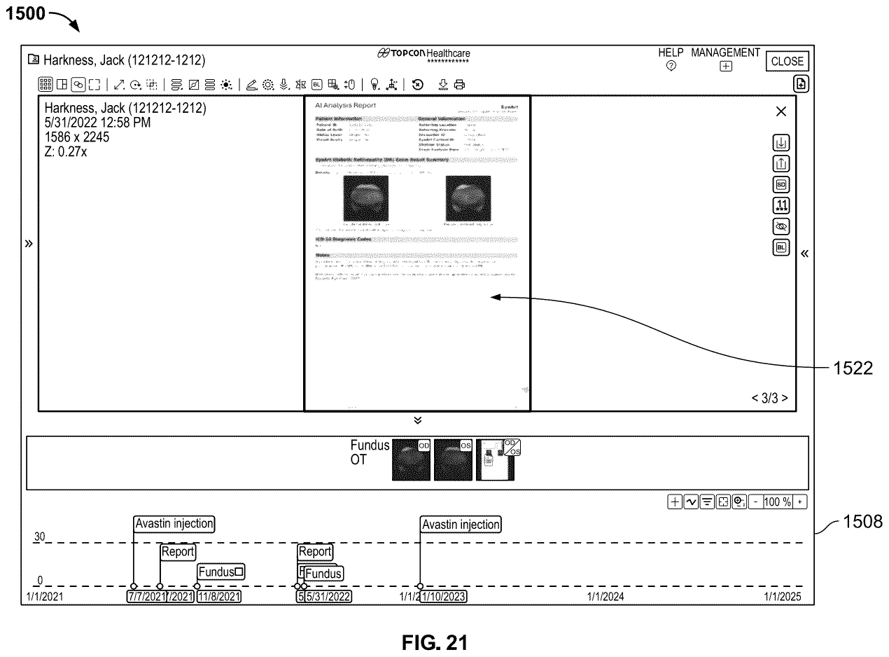

FIG. 20 shows thumbnail 1520 which is associated with the reports generated as indicated in popup window 1518 shown in FIG. 19. FIG. 21 shows report 1522 that was generated based on AI analysis performed as shown in FIGS. 15-20. In one embodiment, an icon associated with report 1522 is shown in timeline 1508.

The foregoing Detailed Description is to be understood as being in every respect illustrative and exemplary, but not restrictive, and the scope of the inventive concept disclosed herein is not to be determined from the Detailed Description, but rather from the claims as interpreted according to the full breadth permitted by the patent laws. It is to be understood that the embodiments shown and described herein are only illustrative of the principles of the inventive concept and that various modifications may be implemented by those skilled in the art without departing from the scope and spirit of the inventive concept. Those skilled in the art could implement various other feature combinations without departing from the scope and spirit of the inventive concept.

1. A method for viewing medical test results in a timeline comprising:
transmitting selected medical test results from a storage device to an AI function in response to activation of the AI function;
receiving an AI analysis report based on the selected medical test results from the AI function;
displaying thumbnails of the selected medical test results on a display;
displaying medical test result icons in a timeline on the display, the medical test result icons representing medical test results; and
displaying an AI analysis report icon on the timeline, the AI analysis report icon representing the AI analysis report.

2. The method of claim 1, wherein the medical test result icons comprise medical test result icons representing selected medical test results and medical test result icons representing unselected medical test results.

3. The method of claim 1, wherein the selected medical test results are automatically selected.

4. The method of claim 1, wherein the selected medical test results are selected by a user.

5. The method of claim 2, wherein the medical test result icons representing selected medical test results are highlighted.

6. The method of claim 3, wherein the selected medical test results are automatically selected based on the selected medical test results being the latest medical test results available.

7. The method of claim 1, further comprising:
displaying a second AI analysis report icon representing a second AI analysis report generated based on second selected medical test results different from the selected medical test results.

8. The method of claim 7, wherein medical test result icons representing the second selected medical test results are highlighted in response to selection of the second AI analysis report icon.

9. An apparatus comprising:
a processor; and
a memory to store computer program instructions, which, when executed on the processor cause the processor to perform operations comprising:
transmitting selected medical test results from a storage device to an AI function in response to activation of the AI function;
receiving an AI analysis report based on the selected medical test results from the AI function;
displaying thumbnails of the selected medical test results on a display;
displaying medical test result icons in a timeline on the display, the medical test result icons representing medical test results; and
displaying an AI analysis report icon on the timeline, the AI analysis report icon representing the AI analysis report.

10. The apparatus of claim 9, wherein the medical test result icons comprise medical test result icons representing selected medical test results and medical test result icons representing unselected medical test results.

11. The apparatus of claim 9, wherein the selected medical test results are automatically selected.

12. The apparatus of claim 9, wherein the selected medical test results are selected by a user.

13. The apparatus of claim 10, wherein the medical test result icons representing selected medical test results are highlighted.

14. The apparatus of claim 9, wherein the selected medical test results are automatically selected based on the selected medical test results being the latest medical test results available.

15. A computer readable medium storing computer program instructions, which, when executed on a processor, cause the processor to perform operations comprising:
transmitting selected medical test results from a storage device to an AI function in response to activation of the AI function;
receiving an AI analysis report based on the selected medical test results from the AI function;
displaying thumbnails of the selected medical test results on a display;
displaying medical test result icons in a timeline on the display, the medical test result icons representing medical test results; and
displaying an AI analysis report icon on the timeline, the AI analysis report icon representing the AI analysis report.

16. The computer readable medium of claim 15, wherein the medical test result icons comprise medical test result icons representing selected medical test results and medical test result icons representing unselected medical test results.

17. The computer readable medium of claim 15, wherein the selected medical test results are automatically selected.

18. The computer readable medium of claim 15, wherein the selected medical test results are selected by a user.

19. The computer readable medium of claim 15, wherein the medical test result icons representing selected medical test results are highlighted.

20. The computer readable medium of claim 16, wherein the selected medical test results are automatically selected based on the selected medical test results being the latest medical test results available.
